(19) United States
(12) Patent Application Publication
(10) Pub. No.: US 2023/0169543 A1
(43) Pub. Date: Jun. 1, 2023

(54) SYSTEM AND METHOD FOR CONTROLLING DISPLAYED CONTENT ON A DIGITAL SIGNAGE

(71) Applicant: USSI Global, Melbourne, FL (US)

(72) Inventors: Theodore H. Korte, Melbourne, FL (US); Anthony M. Morelli, Melbourne, FL (US)

(21) Appl. No.: 17/989,650

(22) Filed: Feb. 22, 2023

Related U.S. Application Data
(60) Provisional application No. 63/280,325, filed on Nov. 17, 2021.

(51) Int. Cl. G06Q 30/0251; B60L 53/30; G06F 3/01

(52) U.S. Cl. CPC G06Q 30/0269; B60L 53/305; G06F 3/011; G06F 2203/011

(57) ABSTRACT

A content management and delivery system for providing targeted content to a user. The system includes a kiosk and sensors for determining whether a user is proximate or within the kiosk and for sensing a user's visually perceptible features.

A storage device stores general content, received primarily from local broadcasters. An experience recommendation engine (AI/ML based) recommends targeted content to a user. The targeted content is selected from the general content based on the emotional state of a current user, as that state is predicted based on the visually perceptible features, and based on a predicted future user behavior or a future user emotional state after exposure to the targeted content. A device at the kiosk presents the targeted content to the user.

### CROSS-REFERENCE TO RELATED APPLICATIONS

The present application claims priority under Section 119(e) to the provisional patent application assigned application No. 63/280,325, filed on Nov. 17, 2021; that provisional application is incorporated herein in its entirety.

### FIELD OF THE INVENTION

The present invention relates to multi-tenant and single-display digital signage and displays, where the content is preferably directed to an individual who is at the signage or in the vicinity of the signage and is selected to engage the individual with the displayed content, where the content is selected based on analysis of the individual's mood, external factors that may influence that mood, and a predicted and desired effect that the content will have on the individual.

### BACKGROUND OF THE INVENTION

Digital signage, electric vehicle (EV) charging stations, and the implementation of smart city ecosystems are concepts that are on a growth trajectory and converging in their respective domains; but they are running in parallel. Businesses across multiple industries are talking about integrating these concepts, but very few have offered successful implementations that merge these new industries into a cohesive approach.

The current state of the art demands a fresh innovative approach that takes advantage of the latest available technologies to provide superior services and products to people. Examples of prior art systems and services include broadcast-feeding of digital signage, targeted advertising, and the use of digital signage for smart cities. But these are all single-use cases. That is, a single content element is decided in advance and then supplied to the digital signage, where it then remains in a static condition until replaced by another static content element.

The single purpose, static signage approach works against the shrinking attention span of shoppers and consumers. New techniques are required to provide optimum value, that is, the right message to the right person at the right time, to engage that person and maintain that engagement for an extended period. Current digital signage is limited to static promoting (advertising) of products and services available from the signage owner. Over time, the single signage approach is repetitive or redundant, loses its value and is not engaging.

This single ownership/continuous operation business model is also very financially limiting for those who have light or only periodic signage needs, or for those who need an affordable solution, such as a small business owner.

Personal mobile phones are the life blood of today's consumers, so any signage, broadcast, or public messaging must coexist and enhance the discovery experiences available on the personal phone. Any attempt to force advertising or provide signage data that is already available on a mobile phone will lose value and likely be ignored. But there is a need for a “First Mover” of information who will support discovery on personal phone applications.

Smart cities require two key elements. First, the collection of data (e.g., by dispersed sensors) that can be used by support organizations, such as law enforcement, emergency services, delivery and pickup services, and city planners. Second, delivery of that useful information to the local population, especially information that offers an avenue for education and inclusion for city residents.

Brick and mortar establishments are under attack from online products and services. The recent pandemic has further pushed consumers out of physical business establishments. Owners are desperately seeking methods to draw people back into their stores and facilities. Digital Signage and targeted ads are one such method.

The traditional retail market has also changed in recent years; merchandising and advertising techniques have become more focused and targeted. To improve advertising conversions, it is crucial to obtain consumer information (e.g., likes, dislikes, acceptable price points) and use that information to target products and services to the consumer. By directing target ads to a consumer who is known to be interested in the advertised product, ad conversion probability is increased.

Today, more information is collected about an individual than ever before and that information is used to personalize advertisements and marketing efforts. Data collection based on internet use is common, as a variety of web analytics collect and analyze user internet behavior, e.g., likes, dislikes, number of clicks on topics, search keywords. But collection of that information is more difficult when the user is not using his computer to conduct business.

Today the essence of retail marketing is all about advertising to the user in small does—often, everywhere, and focused. But traditional linear and forced or fixed advertising is under attack and must transform to be more relevant, contextual, and experience-based. It is well-known to those in the art that relevance-driven advertising yields better results than generalized marketing and merchandising techniques.

Currently, there is no avenue for a multi-tenancy digital signage (that is, multi-purpose and available to diverse audiences). The inventors propose multiple solutions to resolve the issues and problems described above.

### BRIEF DESCRIPTION OF THE FIGURES

The various features of the present inventions will be apparent to one skilled in the art to which the present inventions relate upon consideration of the following description of the invention with reference to the accompanying drawings, herein:

FIG. 1 illustrates the principle components of the system.

FIG. 2 illustrates an exemplary representation of a kiosk.

FIGS. 3A-3C are flowcharts describing the user's interaction with the system of the invention.

FIG. 4 is a flowchart describing operation of the content classification engine.

FIG. 5 is a flowchart describing operation of the behavioral bias classification engine.

FIG. 6 is a flowchart describing operation of the experience recommendation engine.

FIGS. 7A and 7B are terrestrial broadcast related images.

FIG. 8 illustrates a valence/arousal grid.

FIG. 9 is a schematic of neural network elements.

FIG. 10 is a block diagram of a computer system suitable for use with the present invention.

### DETAILED DESCRIPTION OF THE INVENTION

Before describing in detail particular systems and methods for controlling displayed content on a digital signage, it should be observed that the embodiments of the present invention reside primarily in a novel and non-obvious combination of elements and method steps. So as not to obscure the disclosure with details that will be readily apparent to those skilled in the art, certain conventional elements and steps have been presented with lesser detail, while the drawings and the specification describe in greater detail other elements and steps pertinent to understanding the embodiments.

The presented embodiments are not intended to define limits as to the structures, elements or methods of the inventions, but only to provide exemplary constructions. The embodiments are permissive rather than mandatory and illustrative rather than exhaustive.

As will be described in detail below, generally, the system and method of the present invention offer multiple novel and non-obvious features and benefits to provide engaging content to users.

In the following description, for the purposes of explanation, numerous specific details are set forth in order to provide a thorough understanding of the present invention. It will be apparent, however, to one skilled in the art that the present invention may be practiced without these specific details. In other instances, well-known structures and devices are shown in block diagram form in order to avoid unnecessarily obscuring the present invention.

Notwithstanding that the numerical ranges and parameters setting forth the broad scope are approximations, the numerical values set forth in specific non-limiting examples are reported as precisely as possible. Any numerical value, however, inherently contains certain errors necessarily resulting from the standard deviation found in their respective testing measurements at the time of this writing. Furthermore, unless otherwise clear from the context, a numerical value presented herein has an implied precision given by the least significant digit. Thus, a value 1.1 implies a value from 1.05 to 1.15. The term “about” is used to indicate a broader range centered on the given value, and unless otherwise clear from the context implies a broader range around the least significant digit, such as “about 1.1” implies a range from 1.0 to 1.2. If the least significant digit is unclear, then the term “about” implies a factor of two, e.g., “about X” implies a value in the range from 0.5× to 2×, for example, about 100 implies a value in a range from 50 to 200. Moreover, all ranges disclosed herein are to be understood to encompass any and all sub-ranges subsumed therein. For example, a range of “less than 10” for a positive only parameter can include any and all sub-ranges between (and including) the minimum value of zero and the maximum value of 10, that is, any and all sub-ranges having a minimum value of equal to or greater than zero and a maximum value of equal to or less than 10, e.g., 1 to 4.

The advantages of the present invention will be made more apparent from the following description and drawings. It is understood that changes in the specific structure shown and described may be made within the scope of the claims, without departing from the spirit of the invention.

### General System Overview

Generally, the objective of the present invention is to engage a user by presenting specific content (e.g., video, audio, data, images, or photos) to which the user will positively respond. The type of content that will engage the user is dependent both on the mood or present state of mind/emotional state of the user, as well as external conditions that may impact the user's emotional state (e.g., an approaching hurricane).

Sensors collect information that is indicative of the user's current mood or emotional state, such as visually perceptible facial features; a trained neural network receives this information and predicts the users' mood and emotions. Collected information may include, for example, the user's facial characteristics, gestures, facial expressions, and other visually perceptible bodily and facial features.

Relevant external conditions and the mood as determined from the sensor data are input to another trained neural network that predicts the type of content that will engage the user, hopefully moving the user mood to one that causes the user to make a purchase. Many types of content may be engaging, including, for example, learning, entertaining, inspiring, warning, recommending, and marketing, again, depending on the user's emotional state. This “engaging” content is presented to the user.

The system analyzes the sensor-collected data to determine a user's mood or emotional state. The system further determines (using AI/ML concepts and tools) external factors that affect that mood, and how specific content presented to the user will affect that mood. The effect of these three elements on a user can be used to create a first-mover experience. Analyzing the impact that content and external factors have on the user's behavior is critical to place the user in an engaged frame-of-mind. And then offering a product or service to the user and achieve success when the user makes a purchase.

As described herein, the system uses behavioral science concepts, sensor-collected information, the users current or recent past emotional state, and a mix of media content to optimize and personalize the media content presented to the user and thus engage the user.

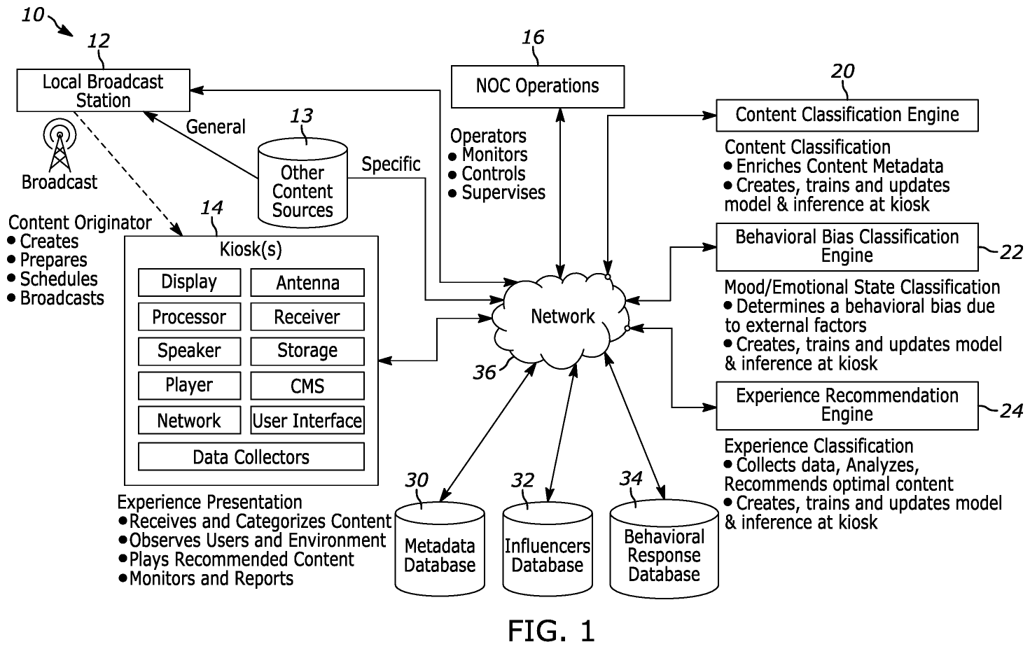

FIG. 1 illustrates the principal components of a digital out-of-home (DOOH) system 10 (one embodiment of the present invention) and the functions performed by each.

The system receives content from a local broadcast station 12, internet sources, and local sources (identified as Other Content Sources 13 in FIG. 1). As a source of readily available content, the local broadcast station serves as the content originator, performing the functions of creating, preparing, scheduling, and broadcasting the content to a kiosk 14. As shown, the content is supplied to a kiosk 14 by an over the air communications link from the local broadcast station 12 and via other known communications systems from the other content sources 13.

The content is provided as multi-format media, including audio, video and graphical, streaming content and static content. Generally, the content can provide a wide range of information that is expected to engage a user, from general inspirational, informational, entertaining, or targeted content, to public service and emergency information.

The content format includes: broadcast clips as files, sensor data, (especially IoT sensor data), messages issued by smart city systems, public safety and public communications, emergency information, broadcast ads, programmatic ads, banner ads, and ads related to local brick-n-mortar businesses.

In addition to providing engaging content to a user, the system serves as an advertising platform for large scale campaigns, such as Coke's Christmas Polar Bear.

The content can also provide information on certain present external conditions that may impact the user's current mood or emotional state. For example, if a hurricane is approaching the location of the user, hurricane updates will clearly engage the user.

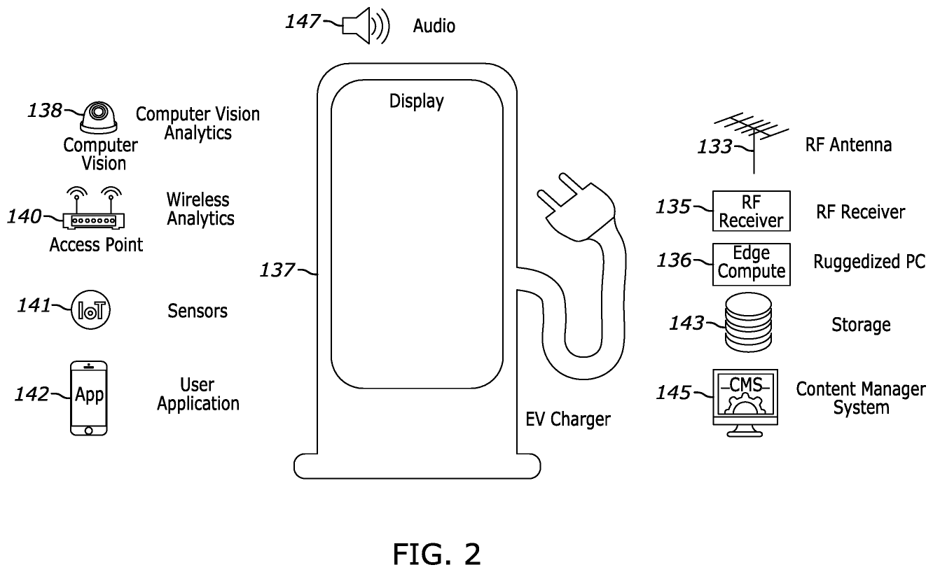

FIG. 1 references the components and functionality at the kiosk 14. Further details of the kiosk are provided in conjunction with FIG. 2. Generally, the kiosk includes sensors (especially IoT sensors) that observe the kiosk users (also sometimes referred to as the audience or as viewers) and those in the vicinity of the kiosk and from the sensor data determines or classifies the user's current mood or emotional state (based on inferences drawn from the sensed data by AI/ML engines to be described below). The system also receives and analyzes media content (also based on AI-based engines) and presents recommended content to the user (also based on an AI-based engine to be described below) that is expected to engage the user.

Although only a single kiosk is depicted in FIG. 1, several kiosks can be connected to a network 18 as controlled by a network controller (NOC) 16. As can be seen, the NOC is the control hub for all networked components. The network operations center monitors, controls, and supervises the network kiosks and all other network components from a centralized location.

In an embodiment with several kiosks, each will concurrently receive the same content within a given broadcast coverage area.

A content classification engine, a behavioral bias classification engine, and an experience recommendation engine are AI-based network elements that receive different inputs and are trained to provide different outputs that ultimately will engage a user at the kiosk. Once trained, the model (also referred to as a inference) created by each of these engines is provided to the local processor (also referred to as an edge computer) at the kiosk for execution.

Content from the local broadcast station 12 and other content sources 13 is input to the kiosk 14 (and stored there) and also supplied to the content classification engine 20 that analyzes and classifies the content. The content class becomes an element of the metadata for the respective content and is stored in a metadata database 30. Classifying or categorizing the content allows the processor at the kiosk to offer (display) content from an appropriate class to the user based on the user's preferences and emotional state, with the ultimate objective of engaging the user.

If the content is of general interest and sent to all kiosks in a broadcast coverage area, the content is referred to as datacasted. If the content is unique to a specific kiosk, the content will typically be supplied through a broadband or internet connection.

After suitable training, this AI-based content classification engine creates and trains a model as to how a particular element of content is expected to affect an emotional state of the user and then hopefully to engage the user. The content classification engine updates the inference engine or model at the edge computer so that the system can present the appropriate content at the appropriate time to the appropriate user. The content classification engine also updates the metadata (as stored in the metadata database 30) of each content element based on effect that element is expected to have on user's emotional state.

As with any AI-based engine, the content classification engine was created and trained using a dataset of content types. In this way a model of each different content type is created, and improved/updated as more content becomes available and is analyzed.

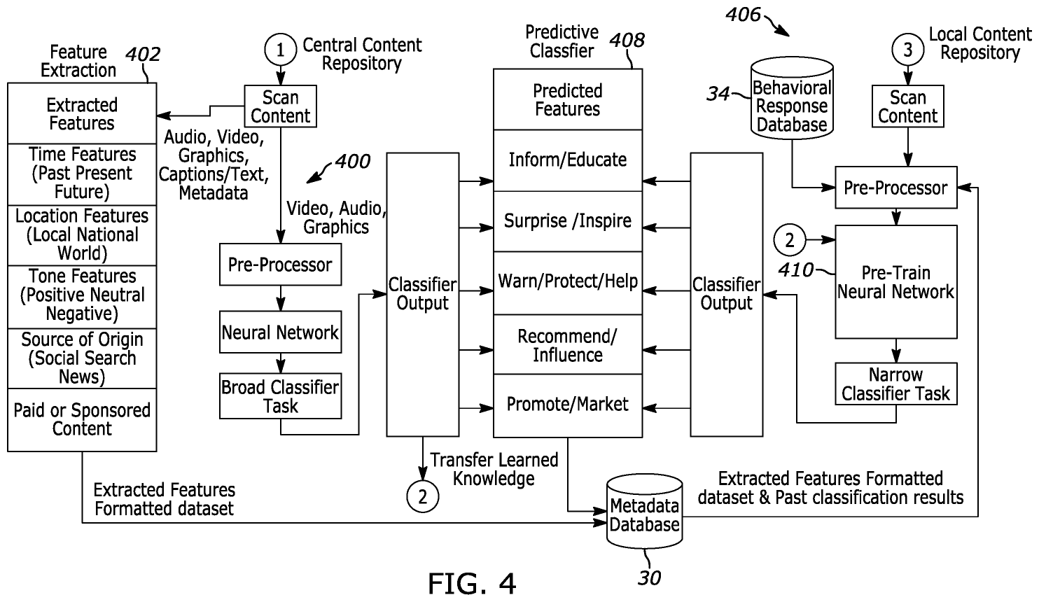

The content classification engine is described in greater detail in conjunction with FIG. 4.

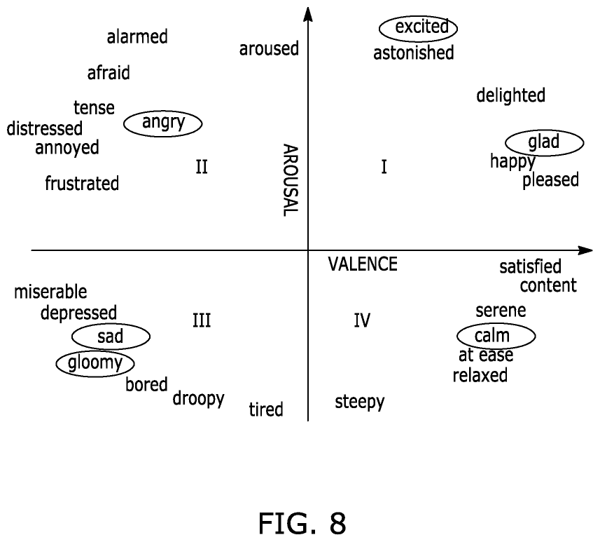

A behavioral bias classification engine 22 predicts the user's mood or emotional state based on external conditions that can affect (a behavioral bias) their emotional state. FIG. 8 depicts possible emotional states of a user. When the system is applied in a retail application, the objective is for the user's mood to be in quadrant I, where the user is most likely to make a purchase when presented with marketing or promotional content.

The behavioral bias classification engine collects and analyzes data related to external conditions or external factors that may influence the user's mood (such as an approaching hurricane). There are many and varied sources from which information regarding these external conditions can be obtained.

Like the content classification engine 20, the behavioral bias classification engine was created and trained using a dataset of different external conditions.

Also, like the content classification engine, models created by the behavioral bias classification engine are executed at the kiosk by the processor/edge computer in a process referred to as inference. And that model must be updated each time the behavioral bias classification engine 22 updates its model. Thus, the reference to updating the model and the inference engine at the kiosk is indicated on FIG. 1.

The experience recommendation engine 24 is also an AI/ML based process that recommends content to be displayed or presented to the user. The presented preferences are based on the predicted mood/emotional state (as determined by the behavioral bias classification engine 22), the expected reaction to different content types (as determined by the content classification engine 20), and user-observed features as determined by sensors at and around the kiosk. Ultimately, the presented content is intended to initiate or maintain the user's engagement at the kiosk. The three AI/ML engines function in concert to identify that optimal content.

The experience recommendation engine also updates the models employed by the edge computer/processor (the inference) at the kiosk to ensure that the optimal content is presented to the user.

According to one embodiment of the system, the content classification engine, the behavioral bias classification engine, and the experience recommendation engine are developed and trained off-site from the kiosk (as depicted in FIG. 1). These engines develop models on which the inferences are based. Each engine supplies the processor at the kiosk data from which to draw appropriate inferences. For example, the class into which a specific content element, received from a broadcaster, should be placed is referred to as an inference. And the predicted effect of a specific external condition on the user's emotional state is another inference determined by the edge computer (based on models supplied behavioral bias classification engine). Thus, as described, there are a number of AI inference models running at the edge computer inside the kiosk.

The metadata database 30 contains descriptive metadata, as provided by the content categorization engine 20, to aid in the classification of content. For example, the content class (e.g., the content is expected to inform/educate or is expected to surprise/inspire) with which a content element has been identified is stored in the metadata database.

An influencer database 32 contains results of the behavioral bias classification engine, that is, external factors (e.g., environmental (e.g., prices, traffic conditions, weather), political, socioeconomic, time-based, seasonal, local, national) that could influence the emotional or behavior state of a user and what is known about their impact on a user's behavior and emotional state. News, social media, sensors, etc. are good sources of these external factors in as close to real-time as possible. Social and psychological research results are good sources of the impact these external factors may have on one's behavior or emotional state.

A behavioral response database 34 stores the stimulus and the user's response to that stimulus (as determined by sensors within the kiosk operating in conjunction with computer vision analytics) from prior presentations of that content. For a particular external stimulus/content, a particular behavioral response (mood) was expected or predicted. But the actual response to that stimulus may have been different than predicted. This information (both as predicted and as experienced) is stored in the behavioral response database 34 and used by the experience recommendation engine to improve the neural network model, i.e., the engine “learns.”

FIG. 2 illustrates an exemplary representation of a kiosk, in one embodiment located at an EV charging station (also referred to as an electric vehicle supply equipment (EVSE). Situating the kiosk at an EV charging station is just one example of the many and varied locations where a kiosk with the described components and functionality can be found.

The kiosk includes multiple devices that can both collect information (sensors, including IoT sensors) about kiosk users and supply information to the kiosk users. In one embodiment, the supplied information can be at the request of a user or as determined by kiosk-based sensors and inference AI/ML-based engines that determine one or more of user actions, attributes, mood, and emotional state, and in response thereto supply relevant ads, information, content, etc. with the intent of engaging the user.

For example, if sensors detect (or the system is informed) that the user drives an electric vehicle that is several years old, the content management system (CMS) displays ads on the digital signage showing current model-year electric vehicles. In this programmatic example, the data collected (e.g., automobile make and model) is provided to an ad-bidding system that offers a real-time ad bidding environment for retailers; an auto dealer sees the bidding opportunity, makes a bid, wins, and supplies the CMS with an automotive ad.

However, generally, the system is typically limited to content that is available locally, e.g., broadcast content that is supplied directly to the kiosk most often by terrestrial broadcast, but also via the internet. If the sensors and data analysis detect a younger professional-looking man who should be in quadrant I (see FIG. 8) and is determined to be in Quadrant I, then the system will typically have a number of ads (content) stored locally to select from. To continue this example, if the man is detected at lunch time and the system content includes an ad for a high-end sandwich restaurant, that ad will be displayed with the intent of engaging him and encouraging him to eat at the sandwich restaurant. Alternatively, the system content may include an ad for a nearby dry-cleaning business, or a promo for a nearby sports bar. The intent is to supply a targeted ad that engages him and encourages him to take his clothes to the dry-cleaning business when he next needs such services and to entice him to return in the evening for a visit to the sports bar. Again, the system objective is to strive for continual engagement by displaying any and all system content that is determined or predicted to be relevant to him.

As also illustrated in FIG. 2, the kiosk includes a digital display system 130 that comprises: IoT sensors 141, including a camera and wireless sensors, an RF antenna 133 operative with a receiver 135 for receiving candidate content (from a local broadcast station, for example) for display on a display 137. A processor 136 (also referred to herein as an edge computer or edge processor) that controls the content management system 145 at the kiosk. The edge computer executes artificial intelligence (AI) inference programs and models that operate the content management system (CMS) and thereby the information displayed. As described elsewhere herein, the inference programs are derived from and updated by the content classification engine 20, the behavioral bias classification engine 22, and the experience recommendation engine 24 (see FIG. 1).

Storage devices 143 store content and media and the AI-based models.

A display 137 displays multi-media content to a user and an audio playback device 147 provides audio-based information to a user. The kiosk also includes a computer vision camera(s) or sensor 138 (operative with a computer vision analytics processing device) and a Wi-Fi access point 140 for use by station users, for example via an application on a smart phone 142. Also, a user can access an internet site via the wireless access point, for example, during a call-to-action engagement event as described elsewhere herein. The access point and app are merely examples, as other interactive devices may be present at the kiosk.

The IoT sensors 141, operating in conjunction with the edge computer 136, can determine the presence of visually perceptible features, attributes, gestures, etc. of the station users, observe the audience surrounding the kiosk, and collect data about the environment proximate the kiosk. Using available AI-based vision-analytics programs, the system can determine gender, age and the emotional state of users. The IoT sensors can provide many different types of information for processing according to various embodiments of the invention and the sensing capabilities of the IoT sensors. For example, sensors for audience, crowd, and vehicular traffic monitoring and control can be used in multiple use cases as described herein. The sensors can also collect vehicular and pedestrian traffic information (such as total count of pedestrians, their direction, speed, etc.) Sensors can also determine user dwell time, i.e., the amount of time the user spends at the display.

Data collected by the following sensors and data collection devices supply information to the decision-making algorithms 20, 22, and 24 of FIG. 1. Since certain of these sensors are not located at the kiosk, a communications link from the sensor to the kiosk and/or to the content management system 145 and/or the edge computer 136 is required. As shown in FIG. 1, that link is provided through a network 36 that links the decision-making algorithms and the kiosk.

Radio frequency identification devices (RFID), near field communications devices (NFC), and QRC (quick response code or simply QR code for scanning by a smart phone) are used to exchange data between the kiosk and users.

Touch screens and automated speech recognition (ASR) devices (not illustrated in FIG. 2) can interact with a user to provide data to or receive data from the user.

Cameras, operating in conjunction with facial recognition software that employs machine learning techniques to compare an acquired image against a database of sample facial expression images, can determine, for example, that a user is smiling. Such facial recognition software can be a component of either or both of the behavioral bias classification engine and the experience recommendation engine of FIG. 1.

Other sensors (not necessarily shown in FIG. 2) supply data to the content management system (CMS) 145 and specifically the various AI/ML engines of FIG. 1, include the following.
A GPS receiver for determining kiosk location data
Biosensors, biometric sensors, electronic sensors, chemical sensors, and smart grid sensors (many of which are used in smart city applications)
Temperature, humidity, sun-index sensors
Vibration sensors (for pot hole detection, for example)
Air quality sensors for determining the air quality index (AQI), UV index, and pollen count
Noise pollution sensors
Light pollution sensors (for example, to detect excess light in regions that are to remain dark to protect certain animal species)
Gun-shot detectors
Waste management sensors
Smart street lights for collecting data from pedestrians
Smart roads for determining road conditions, traffic density, etc.
Wi-Fi access points for providing internet-sourced data
Sensors for determining the QoS (quality of service) of a broadcast signal at the content end-point (i.e., a kiosk), as well as the QoS for internet-supplied data at an internet access point.

The IoT sensors 141 can represent any of the various sensors described herein and others that collect user data and other data for processing by any of the AI/ML-based engines described herein.

As described herein, the collected sensor data is analyzed to identify certain user characteristics and thereby influence the content displayed on the digital signage, as determined by the AI/ML-based engines depicted in FIG. 1.

As can now be appreciated, the present system provides multiple advantages and business opportunities, including:
1. Extends the user experience from a road sign billboard to a parking lot kiosk to an in-store retail purchase experience or to a mobile phone app for an e-commerce experience. For example, the present invention allows a so-called “First Mover” to engage a user then encourage a “Call to Action,” that preferably results in a sale.
2. Provides companion apps, via a proximate Wi-Fi access point, that provides a personalized user experience.
3. Digital signage locations serve as data collection points, including data of value to smart city, city services, and local authorities.
4. Provides a forward-looking user experience by targeting to a user's future needs (as determined from a user profile with survey questions to help target advertising and from real-time sensed data and information about the user) as compared with a user's historical activity.
5. Loyalty programs and gamification
6. Provides other DataCasting use cases

### Content for Presentation to a User

Advantageously, the system generates a unique user experience by using behavioral sciences concepts to present content that is predicted to engage the user. The system also predicts the impact the content is expected to have on the user.

Before the content is provided, system sensors predict the user's mood and as the content is displayed, system sensors also collect information about the individual's reaction to the content. These observations are made in real-time using computer vision analytics and wireless analytics (e.g. Wi-Fi, Bluetooth, ultrasonic, radar, infrared) to anonymously observe activity and behavior of the users near the display.

With this insight, as stored in and supplied by the behavioral response database, the experience recommendation engine 24 suggests the optimum content to engage the individual(s) present at or proximate the display.

Once the individual(s) is engaged, a variety of interactive techniques are used to further extend the user's experience. By analyzing the user's collected behavior data, the CMS causes the digital signage or display component of the system to display content (e.g., audio and/or visual content, or graphical content) to the user with the expectation that the content will engage (or maintain engagement) of the user.

The relevant data can be received or delivered to the engaged user using a Quick Response Code (QRC) or simply QR code, radio frequency identification devices (RFID), and near field communications devices (NFC), voice recognition, touch screen, and other presentation techniques. For example, QRC codes are embedded in the displayed graphics so that the user can collect additional information about the displayed content by scanning to code. Website Uniform Resource Locator (URL) links are provided in the displayed content so that the user can quickly gain access to the website where a product or service can be purchased.

Broadcast content can also be used as a source of information to engage and maintain user engagement. But this broadcast content is to be distinguished from the standard broadcast fare of a traditional linear broadcast stream with embedded commercial breaks. The type content intended for use with the present system is broadcast content delivered live and retained by the system as a file (aka datacasting). Broadcasters produce such professional content several times a day. This content is valuable, but it is also perishable. Breaking News, weather updates, and emergency alerts are examples of high value content when fresh, but content that loses value quickly with time. The system stores a variety of such files with different content. Presentation of such content at the right time to the right user can engage the user. The system controls the type of content to be presented and when, where, how and to whom it is presented, according to the models of the content classification engine 20, the behavioral bias classification engine 22, and the experience recommendation engine 24 (see FIG. 1).

It is instructive to compare the content provided to the user with this invention (e.g., a wide variety of audio, video, and graphics that is frequently refreshed and specifically selected to engage the user), with the content provided by traditional digital signage (i.e., static advertising-only graphics, lacking audio content, that may not be refreshed for several months). The inventive digital signage content can be refreshed much more frequently, in fact as often as desired. Additionally, the availability of video and audio (and tailored video and audio) offers a profound transformation of advertising campaigns.

It is also instructive to compare the content delivery methods of this invention with cellular broadband systems. Broadcast content is the most efficient in terms of audience-reach, as the transfer of one file can reach an unlimited number of individual end points. Because of the efficient one-to-many file delivery system of NextGen broadcast, the content can be changed frequently to maintain contextual relevance to the audience.

The ability to reach a large number of people in a geographical region is important, and a key discriminator of the present invention over cell phone service. During an emergency, every cell user is trying to reach friends and family, send videos, pictures, etc. thus overloading the networks, thereby rendering them slow or completely useless. The system of the invention can target regions within a geographical area to receive the emergency information that pertains to conditions within that region, thereby avoiding a system overload.

A variety of content streams or tracks (referred to as multiple tracks of information or multiple tracks of content) can be presented on the kiosk signage as determined by the AI/ML models in an effort to engage the user. Example content includes:
Ads, offers, and coupons for local brick and mortar businesses
Ads, offers, and coupons targeted to specific viewers/users
Maps showing the location of EV charging stations and access to reservations systems for those stations, including check-in and opt in/out processes. The user must accept the charging station's terms of use and data collection policies due to privacy concerns with data collections and data use. The user also needs an EV charging station account to use the EV charging system (and a Wi-Fi access point if one is available at the charging station). The login/registration process supplements the digital signage data collection process and provides a forward-looking user profile, if the user elects to participate in these additional programs.
Public service ads and data, e.g., smart city conditions, availability of transportation options and highway conditions, e.g., congestion, construction, etc.
The availability and current location of transportation services, such as cars, taxis, trains, metros, etc.
Network and local broadcast programming
Location and availability of emergency services
Surveys and mechanisms to provide feedback
Location and availability of Wi-Fi access points
Personalized companion mobile phone app bearing some relevance to the location of the digital signage, such as an app that allows the user to connect to the kiosk and transition their experience to the website of a retailer at a nearby mall and make a purchase at a discount
Loyalty programs
Social network accesses (including a “Like” button) and gamification access

Terrestrial broadcasting is the most robust and efficient communications channel for reaching large populations instantaneously. When used in conjunction with a local content management system (CMS) (see FIG. 2) of the present invention and a display for presenting content, the content can be targeted for a user (or even for groups of users). The edge processor and CMS control the information displayed, whether the information is intended for large groups of people (weather or traffic information, for example) or smaller groups, such as all owners of electric vehicles (location of nearby charging stations).

Digital signage of the present invention incorporates broadcast information (e.g., the local news, weather and events) and can easily and quickly provide that information to a wide audience using the components and techniques of the present invention.

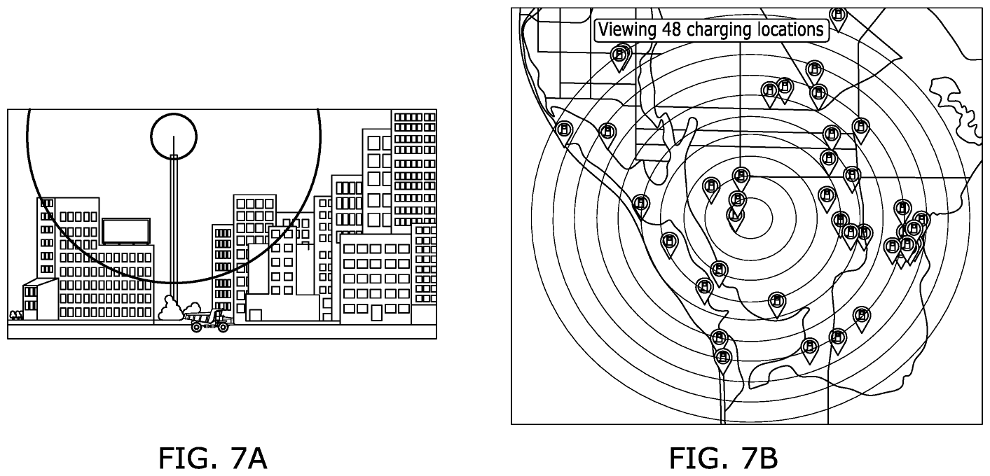

For example, the map of FIG. 7, as displayed by the digital signage system, shows the location of charging stations in a geographical area and also illustrates the one-to-many reach of an over the air (OTA) broadcast signal in the region.

Scenarios using content to engage a user:
Local business advertising alternating with news, weather, environmental conditions, etc. to keep the content engaging and useful to viewers/users as well as those walking near the kiosk.
If the user is engaged with a particular content, she is then presented with subsequent displays related to the engaged content. For example, if the user checks in to the EV charging system, she can pay for the battery charge or the fees are waived if she views ads, including especially ads targeted to her interests. As used herein, engagement refers to an active user who is involved with (engaged with) the presented information. User engagement is more difficult as the user's attention spans declines, such as a user who constantly and passively stares at her mobile phone.
The display can present ads based on the audience of users and conditions sensed by various sensors in the area of the digital signage, as analyzed by the AI/ML engines described relative to FIG. 1.
Emergency information and data is immediately made available and interrupts the presentation of other content.

The digital signage and associated AI/ML systems can be integrated and interface with external ecosystems as follows.
Messaging protocol to identify and target specific signage, such as signage for a central distribution system (a broadcast station or another central content management center).
Messaging protocol used in smart city systems and platforms
Messaging protocol for use with emergency services Advanced Warning and Response Network (AWARN) in the ATSC 3.0 broadcast protocol, Common Alerting Protocol (CAP) in digital signage, etc.
Messaging protocol for use by local vendors to feed information to the digital signage for later (or immediate) display.
Digital Video Ad Serving Template (VAST) protocol with Real-Time Bidding (RTB) Ad Exchange
Interface with electric vehicle apps, for example to obtain information as to the current charge of the EV batteries. This information is useful in determining future EV charging demands.

The information presentation process (that is, what information should be presented to the user to engage them and maintain that engagement) of the invention begins with a desire to influence the needs and wants of the audience, including those people in visual sight of the digital signage, proximate the digital signage, and those who access the digital signage information through a smart phone. By presenting the available content in a manner that is uniquely driven by behavioral analysis (predicted and observed) to bring them from a less desirable state to a more desirable state in which to present promotional content that engages and preferably influences the audience.

The presented information is prioritized to ensure that the most important/immediate information (e.g., emergency conditions) is presented first. Then targeted information is presented to engage the user.

As the information is presented (and before or after the presentation) sensors collect relevant information about those viewing the display for use in identifying relevant information (that is, relevant to the user) and prioritizing the display of future content. In particular, the sensors determine whether the viewer was engaged with the presented information.

It has been determined that requiring user interaction with the digital signage is one technique for maintaining user engagement. Requiring that the user exchange information (referred to as a call-to-action activity) is one technique for ensuring engagement. However, engagement does not necessarily require interaction. Standing in front and enjoying the content is engagement. Interaction is a desired end result to show that the message worked and the user took the next steps.

Interfacing the digital signage with a user's mobile phone also aids user engagement. Upon passing a digital signage, a mobile phone user can “opt-In” from her/his phone and mirror the signage data on the phone display.

Digital signage that is associated with a reservation-based experience (ads for a hotel or restaurant, or display of a map of EV charging stations, for example) permits the user to execute that reservation so that the room, table, or charging stall is available upon arrival.

Of course, for interactive digital signage to be successful and widely used, it must provide attractive, educational, and useful content, in addition to advertising and promotional information. Additionally, the content must be contextually relevant (message, time, location, person viewing, etc.) or initiate or extend information that the user can retrieve from a smart phone accessing internet sites. But, as emphasized herein, the information must be engaging to users.

On-line interactive gaming (especially playing against others) is popular today. Digital signage installations offer another access point for interactive gaming.

Digital signage content can include opportunities to join loyalty programs, including an advertisement associated with the business sponsoring the loyalty program.

In addition to running models to determine targeted content, the edge computer at the location of digital signage or proximate thereto can analyze and report on local environmental conditions through the use of the sensors described herein, such as foot traffic, automobile traffic, weather conditions, available local services, emergency events, etc. As described elsewhere herein, the edge computer also executes models that select and display content with the objective of engaging the user, specifically based on social, psychological, and behavioral sciences, as well as other related scientific findings.

An edge computer that is a component of an EV charging statin can also provide information to potential users, such as the availability of operational stalls, wait times, and available parking at the charging station.

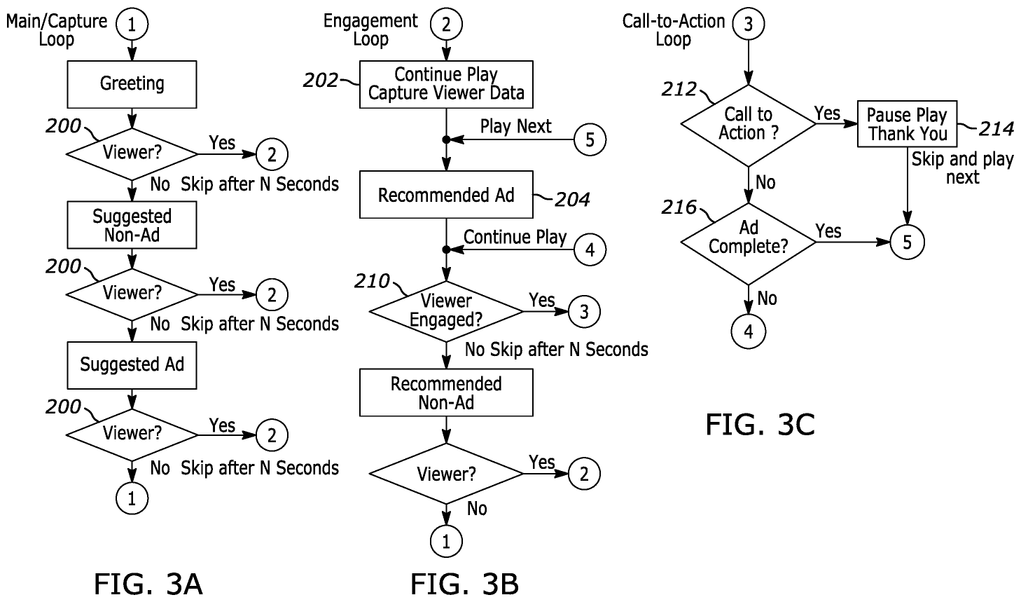

FIGS. 3A, 3B, and 3C depict three coupled software loops related to an encounter with the audience/user.

FIG. 3A depicts the initial encounter with a user (also referred to herein as an audience) during which generic ads or content is presented. The main/capture loop begins by presenting a friendly greeting. Decision blocks 200 indicate that the system continuously checks for a user/viewer. If no viewer is detected, the system plays advertising and non-advertising content based on an AI/ML determined generic-type content recommendation from the experience recommendation engine of FIG. 1. The system terminates playing content when after a predetermined number of seconds if no audience is detected. The main/capture loop does not recommend any specific content, other than showing content that is contextually relevant due to day of the week or a similar generic ad.

If audience is detected at one of the decision blocks 200 the currently playing content plays out in its entirety and then the system jumps to an engagement loop of FIG. 3B.

At step 202 the system continues to play the prior content but also collects viewer data (from sensors located at and proximate the kiosk) for the purpose of determining the viewer's mood or emotional state. The sensors collect data that reflects, number of people, gender, age, mood, emotional state, etc. Once that mood or emotional state is determined by the edge computer at the kiosk (based on the sensor data and the models supplied by the AI/ML-based engines of FIG. 1), a recommended ad or content is presented to the viewer at step 204, with the intent of engaging the user. The recommended ad is based on everything we can predict or observe about the user.

If audience is engaged, as determined at decision block 210, processing jumps to the call-to-action loop in FIG. 3C, which is intended to maintain that engagement. At decision step 212, action by the viewer is encouraged, again, to maintain his/her engagement. For example, the call to action can be a query in an ad for additional viewer information, such as a survey inquiring as the viewer's opinion of multiple car models or cereal brands. Step 214 represents the action by the user. If the system determines that an audience is present and is interacting with the call to action loop, the system pauses to allow ample time for the viewer to interact, with the display. After the interaction is complete, a thank-you message is displayed and execution returns to the engagement loop at call-out 5 (FIG. 3B).

In the event the user refuses the call-to-action invitation, processing continues to step 216. If the prior content has not completed running (a negative decision from decision step 216) an ad or generic content continues to play to its end (call out “4” of the engagement loop, FIG. 3B).

If the running content has been completed, processing continues back to the engagement loop at call out “5” and the next recommended ad or content item is played in an effort to engage (or re-engage the user). If the same user is present at the display, the system displays content similar to the content that initially engaged the user.

After execution of the engagement loop has been completed, whether a user was engaged and the engagement ended or a user was never engaged, processing returns to the main loop of FIG. 3A.

Although not illustrated in FIGS. 3A, 3B, and 3C, the system continuously checks for viewers (users or audience) during execution of the engagement and call to action loops.

At any time during execution of any one of the three loops in FIGS. 3A, 3B, and 3C, processing can be interrupted by an emergency announcement or warning, such as, for example, the approach of a severe thunderstorm. To display this warning, the location of the kiosk must be known for the system to display a relevant warning. The warning is displayed irrespective of whether the system has determined that an audience present at the kiosk.

FIG. 4 illustrates processes for content classification as executed by the content classification engine 20 of FIG. 1.

Both loops 400 and 406 are considered classifier loops. The loop 400 is a broad classifier loop that scans and sorts a very wide range of content or media from a central content repository into categories. The loop 406 learns from the loop 400 (call out “2” Transfer Learned Knowledge), scans the content local to that specific kiosk, and provides a more granular classification (a finer grained classification loop) of the content into one of the specific buckets identified in column 408 of FIG. 4.

In a broad classifier flowchart 400, content is scanned and specific features extracted (see column 402) from that content. The purpose of the broad classifier is to make an initial pass through the content to extract features and thereby create a rough model in which the content is classified into one of the predicted feature classes of column 408.

The content input to the broad classifier loop 400 comprises audio, video, text, captions, and metadata. The extracted features (see column 402) include: time frame of the content (is the content related to a past, present, or future event), location (is the content related to a local, national, or world event), general tone (positive, neutral, or negative), source or origin of the content and whether the content was paid or sponsored (which provides some insight into the motivation and credibility of the content).

In the loop 406, the video, audio, and graphics content is pre-processed where bias values from the extracted features are set and then input to a neural network, which includes a convolutional neural network (CNN) and/or a recursive neural network (RNN). The output of the neural network places each content element into one of the predicted features classes of column 408. For example, content placed into the inform/educate class is expected to inform or educate the user.

As is known by those skilled in the art, a neural network includes both weights and biases to reach a result. Many different types of neural networks have been defined for various uses and more are created every day. Convolutional neural networks are good for image processing (particularly relevant to the present invention) and classification and recursive neural nets are good for language processing (also relevant to the present invention). Certain other neural networks are available and appropriate for use with the present invention, such as those that are designed to classify things. Radial bias feed forward networks (RBFNs) are special types of feedforward neural networks that use radial basis functions as activation functions. Depending on the specific application, known neural networks can be used with the present invention.

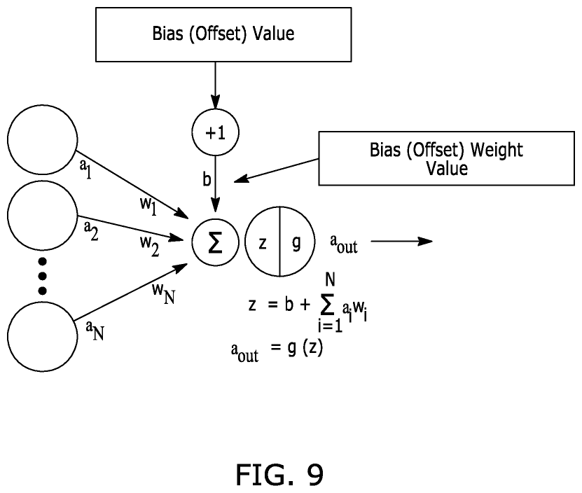

An exemplary neural network is illustrated in FIG. 9. Weights wᵢ control the connection between each neuron by acting as a multiplier for the output of the prior neuron. The product is then input to the next neuron. That is, a weight determines how much influence the input will have on the output. Biases, which are constant, serve as additional inputs to the next layer and always have a value of 1.

Returning to FIG. 4, extracted features formatted dataset are input to the metadata database 30. The transfer learned knowledge data (call out “2”) is input to the pre-train neural network 410 of the classifier flowchart 406. Classifier 406 has a specific task of classifying content into the specified buckets of column 408 and is intended to provide a more accurate classification than the broad classifier of flowchart 400.

Note that the classifier 406 includes feedback from the behavioral response database 34, which provides a history of prior predicted classes and the response of a user when exposed to the content from the predicted class. In other words, the behavioral response database 34 provides a history of the correctly predicted and incorrectly predicted responses of a user.

The classifier flowchart 406 also receives the extracted features determined by the classifier flowchart 400 via the metadata database 30.

The classifier 406 also receives input from the behavioral response database 34. As described elsewhere herein, the database 34 supplies a history of predictions that were intended to engage the user and the result of each prediction, that is, was the prediction accurate.

Once classified, the content can be displayed to the audience based on the intended effect of the presented content (as set forth in column 408) as determined by the experience recommendation engine 24 of FIG. 1.

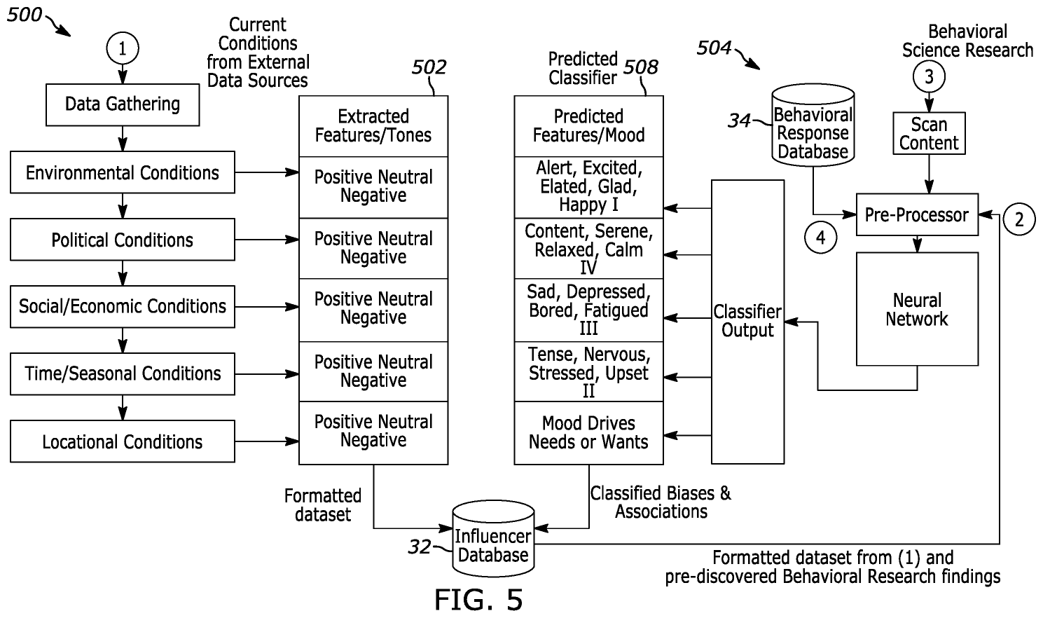

FIG. 5 depicts flowcharts that describe operation of the behavioral bias classification engine 22 of FIG. 1. This engine predicts the mood that the tone of external conditions is expected to evoke in a user.

A flowchart 500 of FIG. 5 depicts a data gathering process that gathers data related to external conditions that may affect a user's mood or emotional state. In one embodiment, the external conditions include: environmental, political, social/economic and time/seasonal conditions and local/location conditions within the region where the kiosk is located. This data is collected from external sources, either online and/or collected locally by sensors proximate the kiosk.

Possible tones for a condition are set forth in column 502. For example, a particular environmental condition within the region is determined to be good, or bad or neutral based on the tone of the content.

In a predictive classifier flowchart 504, the gathered data is input to a neural network, along with inputs from the behavioral response database 34 (which indicates the accuracy of prior predictions), the influencer database 32, and behavioral science concepts.

With reference to the numerals of FIG. 5:
1—Conditions or influences that can be easily gathered from a browser search or social media.
2—A formatted dataset of extracted features/tones as listed in column 502.
3—A formatted dataset of findings gathered from research in behavioral science (for example: more health conscious on Monday, willing to spend more after 6 PM, more likely to impulse buy when sunny, a simple joke can defuse an angry person, etc.).
4—Behavioral Response Database 34 is a dataset of historic results of stimulus response collected from all kiosks

The neural network of FIG. 5 predicts a user's mood or emotional state responsive to the depicted inputs, and then classifies the result into one of the moods of column 508. The Roman numerals in column 508 indicate the related quadrant in the valence/arousal grid of FIG. 8.

The classifier outputs comprise a variety of predicted moods as set forth in column 508. For example, a certain environmental condition may evoke a negative tone, which then causes the classifier to classify the mood associated with that environmental condition as one of tense, nervous, stress, or upset.

Inputs to the influencer database 32 include the formatted dataset from the extracted features/tomes of column 502 and the predicted features/moods from the column 508. The behavior science research input (3) provides an additional dimension to determine if the user is likely to engage in specific types of content. For example, people are more likely to make an impulse buy when the weather is sunny and warm and people are more health conscious on a Monday, specifically when leading into flu season. This type of information is stored in the influencer database 32.

The influencer database 32 provides input to a preprocessor that feeds the neural network.

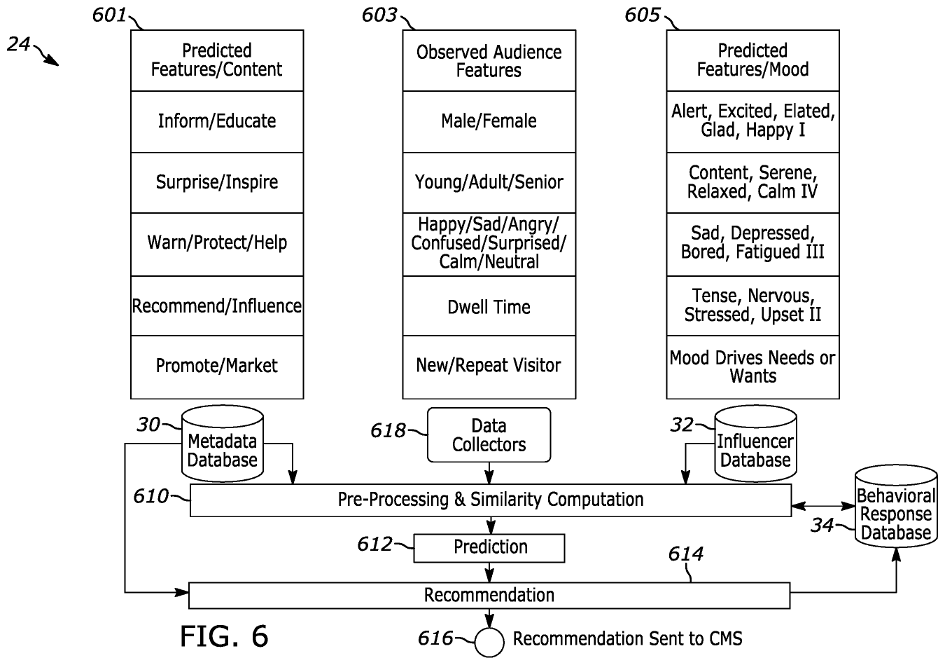

FIG. 6 depicts operation of the experience recommendation engine 24.

Results from the content classification engine are set forth in column 601. The content is stored with the associated enriched metadata (that is, results of the content classification process) in the metadata database 30. This data is input to a pre-processing and similarity computation block 610.
Observed live audience or user features, for users near the display, are listed in column 603. Block 618 indicates that these data collectors supply input to the pre-processing and similarity computation block 610.

Column 605 identifies user moods (and the grid quadrant as depicted in FIG. 8), as determined by the behavioral bias classification engine, based on extracted tones from external conditions. This information is stored in the influencer database 32 and also input to the pre-processing and similarity computation block 610.

Data output from the behavioral response database 34 is also input to the block 610 for identifying similar situations (that is, as related to content, observed audience features, and a predicted mood) and the outcome of those similar situations, where the outcome is based on the visually perceptible features of the user as analyzed by computer vision analytics. These stimulus and response of the predictions and observed behaviors are stored in the database 34 and used to train and update predictive and recommendation models of the prediction block 612 and the recommendation block 614.

The block 610, seeks alignment between the observed mood of the user (as determined by vision analytics performed on the observed user features of column 603) and the predicted mood of the user based on the external conditions of column 605.

Note that the first four lines of column 605 set forth categories or classes of moods as determined by the external conditions of FIG. 5. The fifth line simply refers to a mood that drives needs or wants. Findings gathered from research in behavioral science suggest that people are generally more health conscious on Monday, willing to spend more after 6 PM, more likely to impulse buy when sunny. The arousal/valence grid of FIG. 8 does not necessarily capture these special cases, but these cases may be important to the success of the present invention (e.g., engaging users who make a retail purchase) and therefore represented by the fifth line of column 605.

If the conditions are right and the right opportunity is presented for the right person (albeit not aligned with any of the moods set forth in lines 1-4 of column 605)—that is the holy grail of advertising. The system then sends that right person, the right message at the right time and she makes a purchase.

Based on the data input thereto, the prediction block 612 (another neural network) predicts the type of content that will move the user closer to quadrant I in the FIG. 8 chart. A user with a mood in quadrant I is more likely to make a purchase. And when the system is used in a retail-based embodiment, a user making a purchase is counted as a successful application of the system.

The block 610 first determines whether the conditions are satisfactory for supplying content in the recommending/influencing or the promoting/marketing content class, as this content encourages the user to make a purchase, resulting in revenue for a retailer participating in the system.

If the conditions are not adjudged favorable to make a “sell,” the system attempts to engage and modify the audience behavior. That is, if the user is observed to be bored (after processing the observed audience features of column 603 through the vision analytics engine), but the predictions of column 605 show he should be happy, content that is predicted to surprise/inspire may be displayed. The objective of the presented content is always to move the user's mood to quadrant I of FIG. 8 (which correlates well with the moods listed in the first line of column 605) and then present content that promotes or markets products.

Based on the prediction from the block 612, the recommendation from the block 614 (which uses content and collaborative filtering) is input to the CMS 145 of FIG. 2 for presenting (displaying) the recommended content to the user.

As is known by those skilled in the art, the AI/ML engines described herein are trained before operating in the system of the present invention. During the training process a known dataset of inputs (e.g., content samples for the content classification engine) is supplied to the engine along with a target output (e.g., predicted features of the content, column 408 from FIG. 4 for the content classification engine). Literally thousands of such content samples, the extracted features (column 402) for each sample, and the corresponding predicted feature or target output (column 408) are supplied to train the engine. Each extracted feature is then weighted to indicate the extent to which that extracted feature influences a predicted feature. The weights are a measure of how much that extracted feature influences the predicted feature and the weights are adjusted so that the output of the engine matches the target output for the training dataset. For example, if the training dataset includes content with a local extracted feature that is intended to inform or educate, then a high-value weight is assigned to the local extracted feature.

For standalone digital signage embodiments, i.e., without an IP network connection, the primary system components can include:
EV charger
Digital signage
Current generation or next generation TV antenna and receiver (formally known as ATSC (Advanced Television Systems Committee) 3.0)
Edge processor with media storage
Audience and/or vehicle sensors for determining various characteristics of the audience and/or vehicles
Appropriate IoT sensors
Wi-Fi access point
A phone app, for instance, that uses iBeacon to push notifications via Bluetooth. For example, the kiosk includes a beacon and when someone approaches the kiosk a notification is sent to that person. The notification may be as simple as a welcome note, a local offer/coupon, or a request to check-in for EV charging.

For embodiments that include an IP network or another data channel, the primary components can include:
WAN/IoT network connection. Certain current smart city solutions involve a considerable amount of network data traffic and data analysis, and thus local analysis of the collected data is preferred. Preferably, the data is collected, formatted, and analyzed locally, with only the results (e.g., count, report, summary) sent over the network to a smart city hosting service.
A companion app with a personalization channel for contacting a mobile phone app or other devices on the network. The companion app is a feature of the NextGen broadcast protocol that allows synchronization of broadcast content with secondary data traveling over an IP network. This feature is very helpful to maintain contact and extend or transition the experience from the kiosk to the personal phone. context with the digital signage display. IT is therefore considered a companion to the information or stream presented on the digital signage main screen.
Connections to aggregate data and control across multiple signage locations
Integration of the system components with other systems and devices that are not on the network, for example:
   Ad exchanges
   Smart city information
   Emergency services information
   Local businesses information

### Sensors

The various described embodiments and use cases require the use of sensors (including IoT sensors) and biometric sensors, to determine the emotional state of the user both before the targeted content is provided and after the targeted content is provided (is she still engaged?). These sensors are typically always “on” and observing everything nearby, to assist in determining that emotional state.

In a simple scenario, the profile or emotional state information is collected when the user logs in to a server at the kiosk, when he enters the kiosk based on information gathered by sensors within the kiosk or when he logs in through a mobile phone app. The profile information is then used, as described herein, to select content to display at the kiosk and also content that should not be displayed (perhaps because the user has been determined to have no interest in the subject matter of that content).

It is critical to maintain engagement of users by offering content that the user finds engaging (e.g., interesting, informative, entertaining, etc.). The IoT and biometric sensors (sometimes referred to as audience measurement sensors) collect information and analytics of kiosk users and those walking near the kiosk (likes and dislikes) and based thereon, engaging (hopefully) content is selected by the AI-based inference engines. Such sensors and analysis systems are commercially available from multiple sources,

Sensor collected information that suggests the user is engaged while viewing a particular ad or news story, for example, causes the system to display more related content to maintain the user's engagement.

### Example Use Cases

The techniques and methods of the present invention can be advantageously applied to many different scenarios and use cases, only some of which are described below.

One embodiment of the invention comprises a multi-tenant digital out-of-home (DOOH) signage or display system that is completely self-contained with content supplied by an over the air broadcast. Relevance of the displayed content to the location of the signage and to the user is key, but the content can also be networked with other signage systems and external systems. For example, a widespread linked group of EV chargers throughout a city is a perfect opportunity to combine data collection and communications of relevant content to users across all chargers in the group.

It also desired that the displayed content engage a user/audience of the DOOH according to techniques and the AI/ML engines described herein

The local broadcast station creates the OTA content for transmission to the signage system of the present invention, in particular to each kiosk. Additional content (video, audio, graphics and data) can be created and supplied by other suppliers.

The OTA content includes: conventional broadcast content, content intended for cable and satellite networks, as well as content intended for online distribution. As applied to the present system, the content is in file form so the individual flies can be analyzed and presented to a user based on a number of predicted and observed conditions.

In some cases, the local OTA content can be repurposed for the signage system of the present invention. Specifically, to engage users, the content can be configured with a video clip of interest to the user, concurrently with an informational graphic related to the video clip, and concurrently by a call-to-action, such as a request to the user to take an action that is related to the clip and the graphic. This “call-to-action” step engages the user with the digital signage system by offering one or more interaction opportunities.

In another embodiment, the content is delivered to a digital signage system housed within a kiosk or another sheltered facility. The delivered content includes multiple types of broadcast and online information, such as news, sports, weather, features, entertainment-related information, etc. Upon receipt of the content, the system confirms receipt of the transmission and stores the content. A content management system, including AI-based inference engines, analyzes the content and enriches descriptive metadata with the results of the analysis. An AI-based recommendation engine will later display the content to the user based on the user's emotional state, with the hope of engaging the user. The kiosk, including the system components, is advantageously located in a multi-business area where people tend to gather and spend an extended time.

In yet another use case, a digital road sign attracts vehicular traffic by receiving and displaying interesting content. The content can be supplied by OTA broadcasters such as via an ATSC3 system or broadband internet connection. Piquing her interest, the driver leaves the roadway and parks in a parking lot where the experience continues with digital signage at a parking lot kiosk. Then additional targeted content is displayed at the kiosk. Sensors at the kiosk determine her relevant characteristics that are used in selecting the targeted content that will hopefully keep her engaged.

At the site of the kiosk, the user will also have the opportunity to access a WiFi hotspot to gain Internet access or execute a mobile phone app to extend the interaction experience (a call-to-action) with the digital signage system. Then the engaged experience continues when she enters a nearby retail store that has active shelf displays.

In yet another embodiment the digital signage kiosk is located at an EV charging station. During the charging process the AI-based engines, as described herein, determine the user's emotional state and predict the type of content that will engage the user. As the content is displayed or otherwise presented, proximate IoT and biometric sensors monitor the level of engagement.

Also, in lieu of the user paying directly for the EV charge, the charge is ad-supported with ads from local or national retailers. Loyalty programs can also be used to attract users by awarding points for use of the EV charging system.

In another embodiment, the digital signage can be located at various public venues, or mobile digital signage can be disposed within a taxi, bus, or train.

Imagine driving down city street and seeing a nice bright sign that draws your attention. You look at it and it displays services and products that are available in the nearby strip mall with available parking spots at the mall. You also notice that EV chargers are present in the mall parking lot. Great, you have found a parking spot at an EV charging station with a variety of local retail businesses where you can shop while your vehicle is recharging.

In this example, the EV charging station is the draw-in. Other use cases with different “draw-ins” are within the scope of the present invention. Typically, each use case offers a unique experience (e.g., EV charging, advertising, smart city, brick 'n mortar retailers, broadcast content).

Some of the available services at the location of the “draw-in” range from quick-in and quick-out (e.g., a vape shop) to an hour or two of time (e.g., physical therapy, yoga class, shopping).

Some users may need only an incremental charge and therefore elect to stay in the car during the recharging process. To continue this use case, these users pull up to the charging station for an incremental charge and are given a choice to check-in to use the charger either through an interactive signage kiosk or through an app on their mobile phone. They are also offered a choice to either pay for the charge or have the recharge cost subsidized with ads (local or national in scope) displayed on the proximate signage. The user thus earns a free charge by viewing the presented ads.

In addition to or in lieu of the ads offering a free charge, the system provides informational data (e.g., news, traffic conditions, weather). In a smart city application, the data may include local time, temperature, pollen count, air quality, sun index, weather forecast, upcoming events, road constructions, etc. as collected by smart city sensors. The system may also include a local broadcast option that is available at either the kiosk or on the user's mobile phone through a user-selectable app. The system can also provide, again in lieu of or in addition to the ads, community service information, educational content, and entertainment content. The intent of the presented content is to engage the user and maintain that engagement during the battery charging process. The diversity of information that is available, as described herein, is another value-add element that encourages users to view the digital signage. Additionally, by opting into the mobile phone app, the user can select any content that is available. Ideally, only content that will engage the user is offered. Certainly, the opportunity to select content is likely to keep the user engaged. And if the user has become disengaged, he can easily select different content (information) that will reengage his interest.

A typical restaurant digital signage may offer an idle (static) presentation, such as a menu, lunch specials, etc. If a group of people are awaiting service, the signage system, including appropriate sensors and analysis components, determines who and how many are waiting (e.g., four couples, two families with children, etc.) and then displays interesting or engaging content that is intended to discourage those who are waiting from leaving the restaurant. The objective is to keep the potential customers engaged and therefore not likely to leave before seated. The restaurant digital signage can display multiple messages: the wait time until a table is available, a discount coupon to those waiting, or allow those waiting to order an appetizer or an adult alcoholic beverage.

In another scenario, if an individual is in front of the digital signage and interacting with it, (e.g., watching the local weather report) that individual and the signage can engage in a personalized exchange of data that depends on the type of interaction. For example, the individual can enter her home address for a focused weather report for her neighborhood.

In a smart city ecosystem, multiple datasets are collected, including: type and volume of foot traffic, type and volume of vehicular traffic, and other data that is specifically intended for electric vehicle drivers, such as the location of the nearest charging station. Other examples of collected data in a smart city include monitoring pothole locations, monitoring traffic-light efficiency, parking efficiency etc. Any such data can be displayed in the digital signage of the present invention, either as part of the standard content intended to engage the user or as requested by the user.

In any of the various use cases and embodiments presented, whenever the user appears to be disengaging from the system, the content management system presents different content with the intent of reengaging the user.

Local brick and mortar businesses may want to not only market their business on the digital signage system of the present invention, but also provide coupons at the kiosk or directly to mobile phones to encourage people to become customers of the business. Also, interfacing to the digital signage system and reviewing the information that it collects, offers a convenient means for the business owner to update information regarding their business, links to their website, COVID protocols, etc.

Depending on the venue and the type of engaging content displayed, one use case involves sending user data to an ad exchange for real-time bidding on advertisements to be presented to engaged users. This ad bidding process will likely be an important use case when customers elect an ad-supported EV charging experience and wish to wait in their car while the ads are run on their mobile phone.

Depending on the nature, location, and severity of an emergency event, the digital signage display automatically switches to content that provides emergency services information; advising people the location of the emergency, the location and route of emergency vehicles (so that travel lanes can be cleared), alternative travel routes, expected delay times, etc.

Using appropriately placed sensors, the system can determine the operational status and effectiveness of traffic lights and identify the location of specific vehicles, such as taxis, buses, trains, and emergency vehicles. In the former case, the information can be supplied to the local department of transportation. And in the latter case, these data are supplied to system users, either through digital signage or on a personal mobile phone. These services can also be interactive, such as allowing mobile phone users to search for a taxi or request emergency services. The location, travel route, and ETA of the emergency vehicle can also be shown on the phone display or on the digital signage, such as by illuminating the emergency vehicle location on the phone display.

As described herein, the digital signage system cannot only deliver information but also collect information. For example, AdMobilize computer vision software (available from AdMobilize Software Company of Miami, Fla.) can monitor crowds and provide traffic monitoring. Conventional computer vision uses a camera to detect objects, motion, distance, direction, etc., which may be useful in determining and controlling crowds and traffic.

Other content that can be collected by system sensors then utilized as system content, if only for reporting the information to users, includes: parking space availability, location of street vendors and homeless people, gas prices, time, temperature, air quality, noise pollution, pollen count, wind speed and direction, current traffic information, wait times at retail establishments, an innumerable other datasets that provide valuable information to the citizenry.

The collected data can be correlated with other current similar or historical data for further validation or correlated with other datasets, such as how fast people walk when it is raining vs. sunny vs. cold. How long do people have to wait for a taxi or uber ride under varying conditions such as time of day and weather. Such correlations can be performed by the AI/ML system. These features, which are beyond those available from Google maps, can be easily provided through the digital signage network.

Although several examples and use cases are described in the context of an EV charging station, the described system components can be located at any facility or location where people tend to gather.

### Computer System Description

The embodiments of the present invention may be implemented in the general context of computer-executable instructions, such as program modules executed by a computer. Generally, program modules include routines, programs, objects, components, data structures, etc. that perform particular tasks or implement particular abstract data types. For example, the software programs that underlie the invention can be coded in different languages for use with different platforms. The principles that underlie the invention can be implemented with other types of computer software technologies as well.

Moreover, those skilled in the art will appreciate that the invention may be practiced with other computer system configurations, including hand-held devices, multiprocessor systems, microprocessor-based or programmable consumer electronics, minicomputers, mainframe computers, and the like. The invention may also be practiced in distributed computing environments where tasks are performed by remote processing devices that are linked through a communications network. In a distributed computing environment, program modules may be located in both local and remote computer storage media including memory storage devices.

Persons skilled in the art will recognize that an apparatus, such as a data processing system, including a CPU, memory, I/O, program storage, a connecting bus, and other appropriate components, could be programmed or otherwise designed to facilitate the practice of the method of the invention. Such a system would include appropriate program features for executing the method of the invention.

Also, an article of manufacture, such as a pre-recorded disk or other similar computer program product, for use with a data processing system, could include a storage medium and a program stored thereon for directing the data processing system to facilitate the practice of the method of the invention. Such apparatus and articles of manufacture also fall within the spirit and scope of the invention.

The present invention can be embodied in the form of computer-implemented processes and apparatuses for practicing those processes. The present invention can also be embodied in the form of computer program code containing computer-readable instructions embodied in tangible media, such as floppy diskettes, CD-ROMs, hard disks, flash drives or any other computer-readable storage medium, wherein, when the computer program code is loaded into and executed by a computer or processor, the computer or processor becomes an apparatus for practicing the invention. The present invention can also be embodied in the form of computer program code, for example, whether stored in a storage medium or loaded into and/or executed by a computer, wherein, when the computer program code is loaded into and executed by a computer or processor, the computer or processor becomes an apparatus for practicing the invention. When implemented on a general-purpose computer, the computer program code segments configure the computer to create specific logic circuits or processing modules.

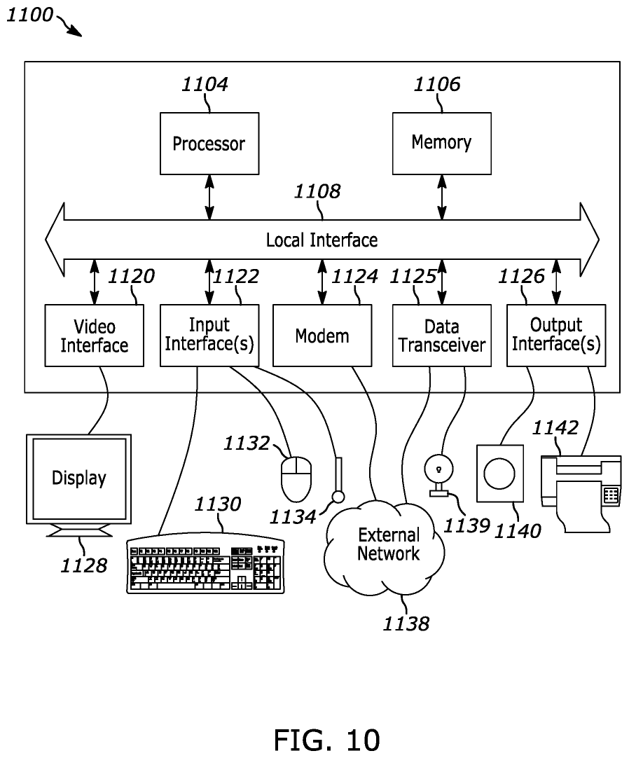

FIG. 10 illustrates a computer system 1100 for use in practicing the invention. The system 1100 can include multiple remotely-located computers and/or processors. The computer system 1100 comprises one or more processors 1104 for executing instructions in the form of computer code to carry out a specified logic routine that implements the teachings of the present invention. The computer system 1100 further comprises a memory 1106 for storing data, software, logic routine instructions, computer programs, files, operating system instructions, and the like, as is well known in the art. The memory 1106 can comprise several devices, for example, volatile and non-volatile memory components further comprising a random access memory RAM, a read only memory ROM, hard disks, floppy disks, compact disks including, but not limited to, CD-ROM, DVD-ROM, and CD-RW, tapes, flash drives and/or other memory components. The system 1100 further comprises associated drives and players for these memory types.

In a multiple computer embodiment, the processor 1104 comprises multiple processors on one or more computer systems linked locally or remotely. According to one embodiment, various tasks associated with the present invention may be segregated so that different tasks can be executed by different computers located locally or remotely from each other.

The processor 1104 and the memory 1106 are coupled to a local interface 1108. The local interface 1108 comprises, for example, a data bus with an accompanying control bus, or a network between a processor and/or processors and/or memory or memories. In various embodiments, the computer system 1100 further comprises a video interface 1120, one or more input interfaces 1122, a modem 1124 and/or a data transceiver interface device 1125. The computer system 1100 further comprises an output interface 1126. The system 1100 further comprises a display 1128. The graphical user interface referred to above may be presented on the display 1128. The system 1100 may further comprise several input devices (not shown) including, but not limited to, a keyboard 1130, a mouse 1131, a microphone 1132, a digital camera and a scanner (the latter two not shown). The data transceiver 1125 interfaces with a hard disk drive 1139 where software programs, including software instructions for implementing the present invention are stored.

The modem 1124 and/or data receiver 1125 can be coupled to an external network 1138 enabling the computer system 1100 to send and receive data signals, voice signals, video signals and the like via the external network 1138 as is well known in the art. The system 1100 also comprises output devices coupled to the output interface 1126, such as an audio speaker 1140, a printer 1142, and the like.

While the invention has been described with reference to preferred embodiments, it will be understood by those skilled in the art that various changes may be made and equivalent elements may be substituted for elements thereof without departing from the scope of the present invention. The scope of the present invention further includes any combination of the elements from the various embodiments as set forth herein. In addition, modifications may be made to adapt the teachings of the present invention to a particular application without departing from its essential scope. Therefore, it is intended that the invention not be limited to the particular embodiment disclosed as the best mode contemplated for carrying out this invention nor to the other embodiments described and/or illustrated, but that the invention will include all embodiments falling within the scope of the appended claims.

Although the subject matter of the invention has been described in relation to specific structural features and/or acts, it is to be understood that the subject matter defined in the appended claims is not necessarily limited to the specific features or acts described above. Rather, the specific features and acts described above are disclosed as example forms of implementing the claims. Accordingly, the invention is not limited except as by the appended claims.

Unless specifically stated otherwise as apparent from the discussion, it is appreciated that throughout the description, discussions utilizing terms such as “processing” or “computing” or “calculating” or “determining” or “displaying” or the like, refer to the action and processes of a computer system, or similar electronic computing device, that manipulates and transforms data represented as physical (electronic) quantities within the computer system's registers and memories into other data similarly represented as physical quantities within the computer system memories or registers or other such information storage, transmission or display devices.

It should be understood that the examples and embodiments described herein are for illustrative purposes only and that various modifications or changes in light thereof will be suggested to persons skilled in the art and are to be included within the spirit and purview of this application.

1. A content management and delivery system for providing targeted content to a user, the system comprising:
a kiosk;
a first sensor at the kiosk for determining whether a user is proximate or within the kiosk;
a second sensor at the kiosk for sensing a user's visually perceptible features;
a storage device for storing general content;
an experience recommendation engine for recommending targeted content, selected from the general content, for the user based on a current user emotional state as predicted from the visually perceptible features, the targeted content intended to achieve a predicted future user behavior or a future user emotional state after exposure to the targeted content; and
a first device at the kiosk for presenting the targeted content to the user.

2. The content management and delivery system of claim 1, further comprising a behavioral response database for supplying the experience recommendation engine effects of historical recommendations issued by the experience recommendation engine, wherein the effects of the historical recommendations are considered by the experience recommendation engine in recommending targeted content.

3. The content management and delivery system of claim 1, wherein the user comprises a user within the kiosk or a user proximate the kiosk.

4. (canceled)

5. The content management and delivery system of claim 1, wherein the visually perceptible features include user gender, appearance, age, facial expressions, gestures, bodily movements, number of users, unique uses, repeat users, attention time, gaze thru rate, and emotion, wherein the visually perceptible features are processed by vision analytics for predicting a user's current emotional state.

6. (canceled)

7. The content management and delivery system of claim 1, wherein the targeted content is intended to engage the user or to influence future user behavior.

8. The content management and delivery system of claim 1, further comprising an influencer database for storing extracted features/tones and predicted features/mood as determined by a behavioral bias classification engine by analyzing external conditions, wherein contents of the influencer database are input to the experience recommendation engine for use in recommending targeted content.

9. The content management and delivery system of claim 1, further comprising a content classification engine for classifying general content based on the influence the general content is predicted to have on the future user behavior or a future user emotional state.

10. The content management and delivery system of claim 1, wherein the content classification engine extracts features from the general content, the features comprising, time features, location features, tone features, content source, trending tags, and paid for or sponsored tags, and wherein the future user behavior or the future user emotional state is further responsive to extracted features, the experience recommendation engine further responsive to extracted features from the general content for use in recommending targeted content.

11. The content management and delivery system of claim 1, wherein a format of the general content comprises video, audio, data, graphical, photographic, image, infographics, call2action, gamification, and loyalty program-related content, wherein the general content is supplied to the kiosk from one or both of broadcast sources and internet-based sources.

12. (canceled)

13. The content management and delivery system of claim 1, wherein the general content comprises one or more of local retailer advertisements, local business information, over-the-air multi-media content, internet-based multimedia content, public service content, public safety content, emergency services information and recommended actions in response thereto, data collected by smart city, smart city communications to residents, and live data streams.

14. The content management and delivery system of claim 1, wherein the future user emotional state comprises an engaged state, and wherein the user is presented with interactive experiences when in the engaged state.

15. The content management and delivery system of claim 14, wherein the user participates in an interactive experience with a smart phone.

16. The content management and delivery system of claim 1, wherein the future user emotional state comprises an engaged state, and wherein while in the engaged state the user is presented with content intended to encourage a purchase by the user.

17. The content management and delivery system of claim 1, further comprising a behavioral bias classification engine for predicting the current user emotional state based on external conditions, wherein the experience recommendation engine recommends targeted content additionally based on a predicted user emotional state based on the external conditions.

18. The content management and delivery system of claim 17, wherein the external conditions are related to environmental, political, social economic, seasonal, time of day, day of week, and locational conditions, and wherein extracted tones associated with each external condition comprise a positive tone, a neutral tone, or a negative tone.

19. (canceled)

20. The content management and delivery system of claim 1, wherein the current user emotional state as predicted from external conditions is described by one of four quadrants on a valence/arousal grid.

21. The content management and delivery system of claim 1, wherein the behavioral bias classification engine employs behavioral science concepts to predict the current user emotional state based on external conditions.

22. The content management and delivery system of claim 1, wherein the first device comprises an audio playback device, a video playback device, or a display.

23. The content management and delivery system of claim 1, wherein a kiosk comprises several kiosks, and wherein a same general content is supplied to each kiosk within a same broadcast coverage area.

24. The content management and delivery system of claim 1, wherein a user can interact with the system using a smart phone by supplying information to the system and receiving information from the system.

25. A method for managing and delivering targeted content to a user at a kiosk, the method comprising:
sensing visually perceptible features of a user at the kiosk;
storing the general content at the kiosk;
using an experience recommendation engine, recommending targeted content, selected from the general content, for the user based on a current user emotional state as predicted from the visually perceptible features, and based on a predicted future user behavior or a future user emotional state after exposure to the targeted content; and
presenting the targeted content to the user.

26. The method for managing and delivering targeted content of claim 25, wherein the visually perceptible features include user gender, appearance, age, facial expressions, gestures, bodily movements, number of users, unique uses, repeat users, attention time, gaze thru rate, and emotion.

27. The method for managing and delivering targeted content of claim 25, wherein the targeted content is intended to engage the user or to influence future user behavior.

28. The method for managing and delivering targeted content of claim 25, further comprising determining and storing extracted features/tones and predicted features/mood by a behavioral bias classification engine analyzing external conditions, and inputting contents of the influencer database to the experience recommendation engine for use in recommending targeted content.

29. The method for managing and delivering targeted content of claim 25, further comprising extracting features from the general content and classifying the general content based on predicted features, extracted features comprising, time features, location features, tone features, content source, trending tags, and paid for or sponsored tags, and inputting extracted features and predicted features to the experience recommendation engine for use in recommending targeted content.

30. The method for managing and delivering targeted content of claim 25, wherein the general content comprises video, audio, data, graphical, photographic, image, infographics, call2action, gamification, and loyalty program-related content, and wherein the general content is supplied to the kiosk from one or both of broadcast sources and internet-based sources.

31. (canceled)

32. The method for managing and delivering targeted content of claim 25, wherein the general content comprises one or more of local retailer advertisements, local business information, over-the-air multi-media content, internet-based multimedia content, public service content, public safety content, emergency services information and recommended actions in response thereto, data collected by smart city, smart city communications to residents, and live data streams.

33. The method for managing and delivering targeted content of claim 25, wherein the future user emotional state comprises an engaged state, the method for managing and delivering targeted content further comprising presenting the user with interactive experiences when in the engaged state.

34. The method for managing and delivering targeted content of claim 25, wherein the future user emotional state comprises an engaged state, the method for managing and delivering targeted content further comprises presenting the user with targeted content intended to encourage a purchase by the user while in the engaged state.

35. The method for managing and delivering targeted content of claim 25, further comprising predicting the current user emotional state based on external conditions by a behavioral bias classification engine, the experience recommendation engine recommending targeted content additionally based on a predicted user emotional state based on the external conditions.

36. The method for managing and delivering targeted content of claim 35, wherein the external conditions are related to environmental, political, social economic, seasonal, time of day, day of week, and locational conditions.

37. The method for managing and delivering targeted content of claim 25, wherein extracted tones associated with each external condition include a positive tone, a neutral tone, or a negative tone.
